(19) United States
(12) Patent Application Publication
(10) Pub. No.: US 2025/0127853 A1
(43) Pub. Date: Apr. 24, 2025

(54) METHODS AND COMPOSITIONS FOR REDUCING NEUTROPHILS OR NEUTROPHIL INFILTRATION AT THE SITE OF DISEASE

(71) Applicant: GNUBIOTICS SCIENCES SA, Epalinges, Lausanne (CH)

(72) Inventor: Adeyemi ADESOKAN, Epalinges (CH)

(73) Assignee: GNUBIOTICS SCIENCES SA, Epalinges, Lausanne (CH)

(21) Appl. No.: 18/689,853

(22) PCT Filed: Sep. 7, 2022
(86) PCT No.: PCT/IB2022/000520

§ 371 Date: Mar. 6, 2024

Related U.S. Application Data
(60) Provisional application No. 63/241,457, filed on Sep. 7, 2021.

(51) Int. Cl. A61K 38/17; A61K 38/01; A61P 37/06

(52) U.S. Cl. CPC A61K 38/1735; A61K 38/012; A61P 37/06

(57) ABSTRACT

Provided herein are methods and compositions for reducing neutrophils and/or neutrophil infiltration at the site of disease using glycopeptide containing compositions.

### CROSS-REFERENCE TO RELATED APPLICATION(S)

This application claims the benefit of U.S. Provisional Application No. 63/241,457, filed Sep. 7, 2021. The entire teachings of the above application are incorporated herein by reference.

### BACKGROUND OF THE INVENTION

Neutrophils are the most abundant circulating leukocytes and are the first line of defense against non-viral infections. However, neutrophils also contribute to tissue damage during various autoimmune and inflammatory diseases. Excessive neutrophils and excessive neutrophil infiltration has been implicated in many diseases and conditions including non-alcoholic fatty liver disease (NAFLD), sepsis, and various lung pathologies. There remains a need to reduce excessive neutrophils and excessive neutrophil infiltration for therapy.

### SUMMARY OF THE INVENTION

Some aspects of the present disclosure are directed to a method of reducing neutrophils or neutrophil infiltration at the site of a disease in a subject in need thereof comprising administering a therapeutic composition comprising a mixture of free glycans and glycopeptides obtained from gastrointestinal tract mucins, wherein the composition is obtained without subjecting the mucins or a partially purified fraction thereof to conditions or reagents that cause complete release of oligosaccharides from glycoproteins or glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the mixture of free glycans and glycopeptides comprises at least one glycopeptide-bound oligosaccharide having a general formulae selected from Hex1HexNAc1, Hex1HexNAc1, Hex1HexNAc1, HexNAc1HexA1, HexNAc1HexA1, HexNAc2, Hex1HexNAc1Sul1, HexNAc1deHex1Sul1, Hex1HexNAc1Sul1, Hex1HexNAc1Sul1, Hex1HexNAc1Sul1, HexNAc2Sul1, HexNAc2Sul1, NeuAc1HexNAc1, NeuAc1HexNAc1, Hex2HexNAc1, Hex1HexNAc1deHex1, Hex1HexNAc1deHex1, Hex1HexNAc1deHex1, Hex1HexNAc1deHex1, Hex1HexNAc1deHex1, Hex1HexNAc1deHex1, Hex2HexNAc1, Hex1HexNAc2, HexNAc2deHex1, Hex1HexNAc2, Hex1HexNAc2, Hex1HexNAc2, Hex1HexNAc1deHex1Sul1, Hex1HexNAc1deHex1Sul1, Hex1HexNAc1deHex1Sul1, Hex1HexNAcSul1, Hex1HexNAcSul1, Hex1HexNAcSul1, Hex2HexNAc1deHex1, NeuAc1Hex1HexNAc1, NeuAc1Hex1HexNAc1, Hex1HexNAc1deHex2, Hex2HexNAc1deHex1, Hex2HexNAc2, Hex1HexNAc2deHex1, Hex1HexNAc2deHex1, Hex1HexNAc2deHex1, Hex1HexNAc2deHex1, Hex1HexNAc2deHex1, Hex2HexNAc2, Hex2HexNAc2, Hex2HexNAc2, Hex2HexNAc2, Hex2HexNAc2, Hex2HexNAc1deHex1Sul1, Hex2HexNAc1deHex1Sul1, Hex1HexNAc1deHex2Sul1, Hex2HexNAc1deHex1Sul1, Hex1HexNAc3, Hex1HexNAc3, Hex1HexNAc2deHex1Sul1, Hex1HexNAc2deHex1Sul1, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, NeuAc1Hex1HexNAc2, Hex1HexNAc2deHex2, Hex2HexNAc2deHex1, Hex2HexNAc2deHex1, Hex2HexNAc2deHex1, Hex2HexNAc2deHex1, Hex2HexNAc2deHex1, Hex1HexNAc3deHex1, Hex1HexNAc3deHex1, Hex2HexNAc3, Hex2HexNAc3, Hex2HexNAc2deHex1Sul1, Hex2HexNAc2deHex1Sul1, Hex2HexNAc2deHex1Sul1, Hex2HexNAc2deHex1Sul1, Hex2HexNAc2deHex1Sul1, Hex1HexNAc4, Hex1HexNAc3deHex1Sul1, Hex1HexNAc3deHex1Sul1, Hex3HexNAc2deHex1, Hex3HexNAc2deHex1, NeuAc1Hex2HexNAc2, Hex2HexNAc2deHex2, Hex2HexNAc3deHex1, Hex2HexNAc3deHex1, Hex2HexNAc3deHex1, Hex3HexNAc2deHex1Sul1, Hex2HexNAc2deHex2Sul1, Hex2HexNAc4, Hex2HexNAc4, Hex2HexNAc3deHex1Sul1, Hex3HexNAc3deHex1, Hex2HexNAc4deHex1, Hex3HexNAc3deHex1Sul1, Hex3HexNAc3deHex1Sul1, and Hex4HexNAc3deHex1Sul1.

In some embodiments, the mixture of free glycans and glycopeptides comprises glycopeptide-bound oligosaccharides having at least 10, at least 20, at least 50, at least 75, or all 99 of the general formulae provided above (e.g., the composition comprises at least 10, 20, 50, 75, or 99 glycopeptides, wherein each of the glycopeptides comprises a different glycopeptide-bound oligosaccharide from the above list).

In some embodiments, the mixture of free glycans and glycopeptides comprises at least one glycopeptide-bound oligosaccharide having a structure selected from Galβ1-3GalNAco1, GlcNAcβ1-4Galo1, GlcNAcα1-4Galo1, HexNAc-GlcAo1, HexNAc-GlcAo1, GlcNAcβ1-6GalNAco1, Galβ1-4(6S)GlcNAco1, 6SGlcNAc-Fuco1, Galβ1-4(6S)GlcNAco1, (S)Galβ1-GlcNAco1, (S)Galβ1-GlcNAco1, 6SGlcNAcβ1-6GalNAco1, 6SGlcNAcβ1-3GalNAco1, NeuAc-HexNAco1, NeuAcα2-6GalNAco1, Galβ1-4GlcNAcβ1-3Gal minus H2O, Fucα1-2(GalNAcα1-3)Galo1, Ga1-GlcNAc-Fuco1, Ga1-GlcNAc-Fuco1, Fucα1-2Galβ1-4GlcNAco1, Fucα1-2Galβ1-3GlcNAco1, Fucα1-2Galβ1-3GalNAco1, Galβ1-4GlcNAcβ1-3Gal, GlcNAcα1-4Galβ1-4GlcNAc minus H2O, GlcNAc-GlcNAc-Fuco1, Galβ1-3(GlcNAcβ1-6)GalNAco1, GlcNAcα1-4Galβ1-3GalNAco1, GlcNAcα1-4Galβ1-4GlcNAco1, Fucα1-2Galβ1-4(6S)GlcNAco1, Galβ1-4(Fucα1-3)(6S)GlcNAco1, 6SGalβ1-3(Fucα1-4)GlcNAco1, SGalβ1-3(GlcNAcβ1-6)GalNAco1, Galβ1-3(6SGlcNAcβ1-6)GalNAco1, Galβ1-4(6S)GlcNAcβ1-6GalNAco1, Fucα1-2Galβ-4GlcNAcβ1-3Gal minus H2O, Galβ1-3(NeuAcα2-6)GalNAco1, NeuAcα2-3Galβ1-3GalNAco1, Fucα1-2Galβ1-4(Fucα1-3)GlcNAco1, Fucα1-2Galβ-4GlcNAcβ1-3Gal, GlcNAcα1-4Galβ1-4GlcNAcβ1-3Gal minus H2O, Fucα1-2(GalNAcα1-3)Galβ1-4GlcNAco1, Fucα1-2(GalNAcα1-3)Galβ1-3GalNAco1, GlcNAcα1-4Gal(Fuc)GlcNAco1, GlcNAcα1-4Gal(Fuc)GlcNAco1, Fucα1-2Galβ1-3(GlcNAcβ1-6)GalNAco1, GlcNAcα1-4Galβ1-4GlcNAcβ1-3Gal, Galβ1-3(Galβ1-4GlcNAcβ1-6)GalNAco1, GlcNAcα1-4Galβ1-4GlcNAcβ1-3Galo1, Galβ1-3(Galβ1-4GlcNAcβ1-6)GalNAco1, Galβ1-4GlcNAcβ1-3Galβ1-3GalNAco1, Fucα1-2Galβ1-4(6S)GlcNAcβ1-4Gal minus H2O, Fucα1-2(S)Galβ1-4GlcNAcβ1-4Gal minus H2O, Fucα1-2Gal(Fuc)(6S)GlcNAco1, Fucα1-2Galβ1-3(6S)GlcNAcβ1-4Galo1, GlcNAcα1-4Galβ1-3(GlcNAcβ1-6)GalNAco1, GlcNAcα1-3Galβ1-4GlcNAcβ1-6GalNAco1, Fucα1-2(GalNAcα1-3)Galβ1-4(6S)GlcNAco1, Fucα1-2Galβ1-4(6S)GlcNAcβ1-3GalNAco1, Fucα1-2Galβ1-3(6S-GlcNAcβ1-6)GalNAco1, Fucα1-2Galβ1-3(NeuAcα2-6)GalNAco1, Galβ1-3[Galβ1-4(6S)GlcNAcβ1-6]GalNAco1, GlcNAcα1-4Galβ1-3(NeuAcα2-6)GalNAco1, GlcNAcα1-4Galβ1-3[(6S)GlcNAcβ1-6]GalNAco1, GalNAcα1-3(Fucα1-2)Galβ1-4GlcNAcβ1-3Gal minus H2O, GlcNAcα1-4Galβ1-3(NeuAcα2-6)GalNAco1, Fucα1-2(GalNAcα1-3)Ga1-(Fuc)GlcNAco1, Galβ1-3(Fucα1-4)GlcNAcβ1-3Galβ1-3GalNAco1, Galβ1-4(Fucα1-3)GlcNAcβ1-3Galβ1-3GalNAco1, Fucα1-2Galβ1-4GlcNAcβ1-3Galβ1-4GlcNAco1, Galβ1-3(Fucα1-2Galβ1-4GlcNAcβ1-6)GalNAco1, Fucα1-2Galβ1-3(Galβ1-4GlcNAcβ1-6)GalNAco1, Fucα1-2(GalNAcα1-3)Galβ1-4GlcNAcβ1-6GalNAco1, Fucα1-2Galβ1-3(GalNAcβ1-4GlcNAcβ1-6)GalNAco1, Galβ1-4GlcNAcβ1-3(GlcNAcβ1-6)Galβ1-3GalNAco1, GlcNAcα1-4Galβ1-3(Galβ1-4GlcNAcβ1-6)GalNAco1, GalNAcα1-3(Fucα1-2)Galβ1-4(6S)GlcNAcβ1-3Gal minus H2O, Fucα1-2Galβ1-4(6S)GlcNAcβ1-3Galβ1-3GalNAco1, Fucα1-2Galβ1-3[Galβ1-4(6S)GlcNAcβ1-6]GalNAco1, GlcNAcα1-4Galβ1-3(GalNAcβ1-4GlcNAcβ1-6)GalNAco1, GlcNAcβ1-3[Fucα1-2Galβ1-4(6S)GlcNAcβ1-6]GalNAco1, GalNAcα1-3(Fucα1-2)Galβ1-3(6SGlcNAcβ1-6)GalNAco1, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAco1, Fucα1-2Galβ1-3(Fucα1-2Galβ1-4GlcNAcβ1-6)GalNAco1, Galβ1-3[Fucα1-2(GalNAcα1-3)Galβ1-4GlcNAcβ1-6]GalNAco1, GlcNAcα1-4Galβ1-3(Fucα1-2Galβ1-4GlcNAcβ1-6)GalNAco1, Fucα1-2Galβ1-3(GlcNAcα1-4Galβ1-4GlcNAcβ1-6)GalNAco1, Fucα1-2Galβ1-4(6S)GlcNAcβ1-3(Galβ1-4GlcNAcβ1-6)Gal minus H2O, Fucα1-2Galβ1-3[Fucα1-2Galβ1-4(6S)GlcNAcβ1-6]GalNAco1, GlcNAcα1-4Galβ1-3(GlcNAcα1-4Galβ1-4GlcNAcβ1-6)GalNAco1, Ga1-GlcNAcβ1-3(GlcNAcα1-4Galβ1-4GlcNAcβ1-6)GalNAco1, GalNAcα1-3(Fucα1-2)Galβ1-4(6S)GcNAcβ1-3Galβ1-3GalNAco1, Fucα1-2Galβ1-4GlcNAcβ1-3Galβ1-4GlcNAcβ1-3Galβ1-3GalNAco1, GlcNAcα1-4Galβ1-3[GalNAcα1-3(Fucα1-2)Galβ1-4GlcNAcβ1-6]GalNAco1, linear structure, Fucα1-2Galβ1-4(6S)GlcNAcβ1-3Galβ1-4GlcNAcβ1-3Galβ1-4GlcNAco1, and Fucα1-2Galβ1-4(6S)GlcNAcβ1-3Galβ1-4GlcNAcβ1-3(Galβ1-4GlcNAcβ1-6)Gal minus H2O.

In some embodiments, the mixture of free glycans and glycopeptides comprises glycopeptide-bound oligosaccharides having at least 10, at least 20, at least 50, at least 75, or all 99 of the structures provided above (e.g., the composition comprises at least 10, 20, 50, 75, or 99 glycopeptides, wherein each of the glycopeptides comprises a different glycopeptide-bound oligosaccharide from the above list).

In some embodiments, the therapeutic composition has a salt content of less than about 2%. In some embodiments, the therapeutic composition is a powder. In some embodiments, the therapeutic composition has a free glycan content of less than 0.1% by weight.

In some embodiments, the disease is selected from the group consisting of excessive neutrophil infiltration of an organ or tissue, excessive neutrophil activation, acute lung injury, sepsis, ischemia-reperfusion injury, myocardial infarction, stroke, nonalcoholic fatty liver disease (NAFLD), metabolic-associated fatty liver disease (MAFLD), pleurisy, pulmonary fibrosis, systemic sclerosis, chronic obstructive pulmonary disease (COPD), adult respiratory response syndrome (ARDS), peripheral blood neutrophilia, systemic inflammatory response syndrome (SIRS), derangement of coagulation, fibrinolysis, disseminated intravascular coagulation, coagulopathy, vasculitis, or neutrophil-mediated tissue damage and remodelling.

In some embodiments, the therapeutic composition is administered orally or rectally. In some embodiments, the therapeutic composition further comprises one or more excipients or carriers.

Some aspects of the present disclosure are directed to a method of manufacturing a therapeutic composition disclosed herein, comprising the following steps: hydrolyzing an aqueous solution containing mucin powder with Ca(OH)2 at 60° C. for about 3 hours; cooling the solution to room temperature, adding diatomaceous earth, filtering the solution and collecting the filtrate; neutralizing the filtrate to a pH of 7.5 with CO2 and, optionally, adding diatomaceous earth, filtering and collecting the filtrate; contacting the filtrate with Dowex ion exchange hydrogen form resin to adjust the pH to 6.5 to 7, filtering and collecting the filtrate; and concentrating and spray drying the filtrate to obtain a powder. In some embodiments, the therapeutic composition is manufactured by a method comprising the method provided in Example 1 or 2 below.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows that GBX administration reduces the disease activity score caused by DSS (Dextran sulfate sodium) administration.

FIG. 2 shows that GBX administration reduces elevated neutrophil levels caused by DSS (Dextran sulfate sodium) administration.

FIG. 3 shows the general formulae and putative structures of glycopeptide-bound oligosaccharide present in the GBX preparation of Example 1.

### DETAILED DESCRIPTION OF THE INVENTION

### Some Definitions

The expression “gastrointestinal tract mucins” encompasses any natural source of mucin from which glycans and glycopetides can be extracted, suitable for mammalian nutrition or pharmaceutical use. Typical sources of gastrointestinal tract mucins are extracts from gastrointestinal tract, in particular from porcine sources or from bovine sources. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China). In some embodiments, the gastrointestinal tract mucins are from porcine gastric mucus or pig slime. “Pig slime” is mucus extracted from pig stomachs, usually in slaughterhouses, by extraction of the upper layer of the inner stomach tissue.

The expression “subject” refers to mammals. For examples, mammals contemplated by the present invention include human, primates, domesticated animals such as cattle, sheep, pigs, horses, rodents, cats, dogs and other pets. In some embodiments, the subject is a human. In some embodiments, the subject may be an infant (1 year old or less for a human), a toddler (3 years old or less for a human), a child, a young adult, an adult or a geriatric. In some embodiments, the infant is a newborn. The subject may be male or female. In some embodiments, the subject is female and of child-bearing age.

The term “glycoprotein” refers to proteins linked to oligosaccharides, e.g. proteins either N-linked or O-linked to oligosaccharides, and having a molecular weight of more than about 5 KDa.

The term “glycopeptide” refers to peptides linked to oligosaccharides, e.g. peptides either N-linked or O-linked to oligosaccharides, and having a molecular weight of less than about 5 KDa. Methods of determining molecular weight of glycopeptides and glycoproteins are known in the art and are not limited. In some embodiments, the molecular weight of glycopeptides and glycoproteins are determined by size exclusion chromatography.

The term “glycan” as used herein refers to an oligosaccharide that has been released or removed from a glycoprotein or glycopeptide. The term is synonymous with the term “free glycan” and “free oligosaccharide,” as also used herein.

In some embodiments, peptides are defined as having a molecular weight of less than about 5 KDa. In some embodiments, the term peptides include glycopeptides. In some embodiments, proteins are defined as having a molecular weight of more than about 5 KDa. In some embodiments, the term proteins include glycoproteins.

As used herein, “a partially purified fraction” of gastrointestinal tract mucins comprises at least about 40%, at least about 50%, at least about 60%, at least about 70%, at least about 80%, at least about 90%, at least about 92.5%, at least about 95%, at least about 97.5%, at least about 98%, at least about 99%, or at least about 99.5% of the protein- and peptide-glycans present in un-purified gastrointestinal tract mucins. In some embodiments, the mucins or partially purified fraction thereof has been subject to an acid treatment.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Reducing Neutrophils or Neutrophil Infiltration at the Site of a Disease

Some aspects of the present disclosure are directed to a method of reducing neutrophils or neutrophil infiltration at the site of a disease in a subject in need thereof comprising administering a therapeutic composition comprising a mixture of free glycans and glycopeptides obtained from gastrointestinal tract mucins, wherein the composition is obtained without subjecting the mucins or a partially purified fraction thereof to conditions or reagents that cause complete release of oligosaccharides from glycoproteins or glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the total oligosaccharide content of the therapeutic composition (e.g., composition) is greater than about 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the total oligosaccharide content is the total weight of oligosaccharide in the composition. Such weight does not include the weight of proteins or peptides attached to the oligosaccharides. In some embodiments, the oligosaccharide content of the composition is greater than about 10% (w/w). In some embodiments, the oligosaccharide content of the composition is greater than or equal to about 15% (w/w). In some embodiments, the oligosaccharide content of the composition is greater than or equal to about 20% (w/w). In some embodiments, the oligosaccharide content comprises substantially all oligosaccharides bound to glycoprotein or glycopeptide without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the ratio of glycopeptides:free glycans is >4:1 (w/w). In some embodiments, the ratio of glycopeptides:free glycans (w/w) is about 4:1, about 5:1, about 6:1, about 7:1, about 8:1, about 9:1, about 10:1, about 11:1, about 12:1, about 13:1, about 14:1, about 15:1, about 20:1, or more.

In some embodiments, the total glycoprotein content of the composition is 12% or less (w/w). In some embodiments, the total glycoprotein content of the composition is 11%, 10%, 9%, 8%, 7%, 6%, 5%, 4%, 3%, 2%, 1%, or less (w/w).

In some embodiments, the mixture of free glycans and glycopeptides comprises at least one glycopeptide-bound oligosaccharide having a general formulae selected from Hex1HexNAc1, Hex1HexNAc1, Hex1HexNAc1, HexNAc1HexA1, HexNAc1HexA1, HexNAc2, Hex1HexNAc1Sul1, HexNAc1deHex1Sul1, Hex1HexNAc1Sul1, Hex1HexNAc1Sul1, Hex1HexNAc1Sul1, HexNAc2Sul1, HexNAc2Sul1, NeuAc1HexNAc1, NeuAc1HexNAc1, Hex2HexNAc1, Hex1HexNAc1deHex1, Hex1HexNAc1deHex1, Hex1HexNAc1deHex1, Hex1HexNAc1deHex1, Hex1HexNAc1deHex1, Hex1HexNAc1deHex1, Hex2HexNAc1, Hex1HexNAc2, HexNAc2deHex1, Hex1HexNAc2, Hex1HexNAc2, Hex1HexNAc2, Hex1HexNAc1deHex1Sul1, Hex1HexNAc1deHex1Sul1, Hex1HexNAc1deHex1Sul1, Hex1HexNAcSul1, Hex1HexNAcSul1, Hex1HexNAcSul1, Hex2HexNAc1deHex1, NeuAc1Hex1HexNAc1, NeuAc1Hex1HexNAc1, Hex1HexNAc1deHex2, Hex2HexNAc1deHex1, Hex2HexNAc2, Hex1HexNAc2deHex1, Hex1HexNAc2deHex1, Hex1HexNAc2deHex1, Hex1HexNAc2deHex1, Hex1HexNAc2deHex1, Hex2HexNAc2, Hex2HexNAc2, Hex2HexNAc2, Hex2HexNAc2, Hex2HexNAc2, Hex2HexNAc1deHex1Sul1, Hex2HexNAc1deHex1Sul1, Hex1HexNAc1deHex2Sul1, Hex2HexNAc1deHex1Sul1, Hex1HexNAc3, Hex1HexNAc3, Hex1HexNAc2deHex1Sul1, Hex1HexNAc2deHex1Sul1, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, NeuAc1Hex1HexNAc2, Hex1HexNAc2deHex2, Hex2HexNAc2deHex1, Hex2HexNAc2deHex1, Hex2HexNAc2deHex1, Hex2HexNAc2deHex1, Hex2HexNAc2deHex1, Hex1HexNAc3deHex1, Hex1HexNAc3deHex1, Hex2HexNAc3, Hex2HexNAc3, Hex2HexNAc2deHex1Sul1, Hex2HexNAc2deHex1Sul1, Hex2HexNAc2deHex1Sul1, Hex2HexNAc2deHex1Sul1, Hex2HexNAc2deHex1Sul1, Hex1HexNAc4, Hex1HexNAc3deHex1Sul1, Hex1HexNAc3deHex1Sul1, Hex3HexNAc2deHex1, Hex3HexNAc2deHex1, NeuAc1Hex2HexNAc2, Hex2HexNAc2deHex2, Hex2HexNAc3deHex1, Hex2HexNAc3deHex1, Hex2HexNAc3deHex1, Hex3HexNAc2deHex1Sul1, Hex2HexNAc2deHex2Sul1, Hex2HexNAc4, Hex2HexNAc4, Hex2HexNAc3deHex1Sul1, Hex3HexNAc3deHex1, Hex2HexNAc4deHex1, Hex3HexNAc3deHex1Sul1, Hex3HexNAc3deHex1Sul1, and Hex4HexNAc3deHex1Sul1.

In some embodiments, the mixture of free glycans and glycopeptides comprises glycopeptide-bound oligosaccharides having at least 10, at least 20, at least 50, at least 75, or all 99 of the general formulae provided above (e.g., the composition comprises at least 10, 20, 50, 75, or 99 glycopeptides, wherein each of the glycopeptides comprises a different glycopeptide-bound oligosaccharide from the above list). In some embodiments, the mixture of free glycans and glycopeptides comprises glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all 99 of the general formulae provided above.

In some embodiments, the mixture of free glycans and glycopeptides comprises at least one glycopeptide-bound oligosaccharide having a structure selected from Galβ1-3GalNAco1, GlcNAcβ1-4Galo1, GlcNAcα1-4Galo1, HexNAc-GlcAo1, HexNAc-GlcAo1, GlcNAcβ1-6GalNAco1, Galβ1-4(6S)GlcNAco1, 6SGlcNAc-Fuco1, Galβ1-4(6S)GlcNAco1, (S)Galβ1-GlcNAco1, (S)Galβ1-GlcNAco1, 6SGlcNAcβ1-6GalNAco1, 6SGlcNAcβ1-3GalNAco1, NeuAc-HexNAco1, NeuAcα2-6GalNAco1, Galβ1-4GlcNAcβ1-3Gal minus H2O, Fucα1-2(GalNAcα1-3)Galo1, Ga1-GlcNAc-Fuco1, Ga1-GlcNAc-Fuco1, Fucα1-2Galβ1-4GlcNAco1, Fucα1-2Galβ1-3GlcNAco1, Fucα1-2Galβ1-3GalNAco1, Galβ1-4GlcNAcβ1-3Gal, GlcNAcα1-4Galβ1-4GlcNAc minus H2O, GlcNAc-GlcNAc-Fuco1, Galβ1-3(GlcNAcβ1-6)GalNAco1, GlcNAcα1-4Galβ1-3GalNAco1, GlcNAcα1-4Galβ1-4GlcNAco1, Fucα1-2Galβ1-4(6S)GlcNAco1, Galβ1-4(Fucα1-3)(6S)GlcNAco1, 6SGalβ1-3(Fucα1-4)GlcNAco1, SGalβ1-3(GlcNAcβ1-6)GalNAco1, Galβ1-3(6SGlcNAcβ1-6)GalNAco1, Galβ1-4(6S)GlcNAcβ1-6GalNAco1, Fucα1-2Galβ-4GlcNAcβ1-3Gal minus H2O, Galβ1-3(NeuAcα2-6)GalNAco1, NeuAcα2-3Galβ1-3GalNAco1, Fucα1-2Galβ1-4(Fucα1-3)GlcNAco1, Fucα1-2Galβ-4GlcNAcβ1-3Gal, GlcNAcα1-4Galβ1-4GlcNAcβ1-3Gal minus H2O, Fucα1-2(GalNAcα1-3)Galβ1-4GlcNAco1, Fucα1-2(GalNAcα1-3)Galβ1-3GalNAco1, GlcNAcα1-4Gal(Fuc)GlcNAco1, GlcNAcα1-4Gal(Fuc)GlcNAco1, Fucα1-2Galβ1-3(GlcNAcβ1-6)GalNAco1, GlcNAcα1-4Galβ1-4GlcNAcβ1-3Gal, Galβ1-3(Galβ1-4GlcNAcβ1-6)GalNAco1, GlcNAcα1-4Galβ1-4GlcNAcβ1-3Galo1, Galβ1-3(Galβ1-4GlcNAcβ1-6)GalNAco1, Galβ1-4GlcNAcβ1-3Galβ1-3GalNAco1, Fucα1-2Galβ1-4(6S)GlcNAcβ1-4Gal minus H2O, Fucα1-2(S)Galβ1-4GlcNAcβ1-4Gal minus H2O, Fucα1-2Gal(Fuc)(6S)GlcNAco1, Fucα1-2Galβ1-3(6S)GlcNAcβ1-4Galo1, GlcNAcα1-4Galβ1-3(GlcNAcβ1-6)GalNAco1, GlcNAcα1-3Galβ1-4GlcNAcβ1-6GalNAco1, Fucα1-2(GalNAcα1-3)Galβ1-4(6S)GlcNAco1, Fucα1-2Galβ1-4(6S)GlcNAcβ1-3GalNAco1, Fucα1-2Galβ1-3(6S-GlcNAcβ1-6)GalNAco1, Fucα1-2Galβ1-3(NeuAcα2-6)GalNAco1, Galβ1-3[Galβ1-4(6S)GlcNAcβ1-6]GalNAco1, GlcNAcα1-4Galβ1-3(NeuAcα2-6)GalNAco1, GlcNAcα1-4Galβ1-3[(6S)GlcNAcβ1-6]GalNAco1, GalNAcα1-3(Fucα1-2)Galβ1-4GlcNAcβ1-3Gal minus H2O, GlcNAcα1-4Galβ1-3(NeuAcα2-6)GalNAco1, Fucα1-2(GalNAcα1-3)Ga1-(Fuc)GlcNAco1, Galβ1-3(Fucα1-4)GlcNAcβ1-3Galβ1-3GalNAco1, Galβ1-4(Fucα1-3)GlcNAcβ1-3Galβ1-3GalNAco1, Fucα1-2Galβ1-4GlcNAcβ1-3Galβ1-4GlcNAco1, Galβ1-3(Fucα1-2Galβ1-4GlcNAcβ1-6)GalNAco1, Fucα1-2Galβ1-3(Galβ1-4GlcNAcβ1-6)GalNAco1, Fucα1-2(GalNAcα1-3)Galβ1-4GlcNAcβ1-6GalNAco1, Fucα1-2Galβ1-3(GalNAcβ1-4GlcNAcβ1-6)GalNAco1, Galβ1-4GlcNAcβ1-3(GlcNAcβ1-6)Galβ1-3GalNAco1, GlcNAcα1-4Galβ1-3(Galβ1-4GlcNAcβ1-6)GalNAco1, GalNAcα1-3(Fucα1-2)Galβ1-4(6S)GlcNAcβ1-3Gal minus H2O, Fucα1-2Galβ1-4(6S)GlcNAcβ1-3Galβ1-3GalNAco1, Fucα1-2Galβ1-3[Galβ1-4(6S)GlcNAcβ1-6]GalNAco1, GlcNAcα1-4Galβ1-3(GalNAcβ1-4GlcNAcβ1-6)GalNAco1, GlcNAcβ1-3[Fucα1-2Galβ1-4(6S)GlcNAcβ1-6]GalNAco1, GalNAcα1-3(Fucα1-2)Galβ1-3(6SGlcNAcβ1-6)GalNAco1, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAco1, Fucα1-2Galβ1-3(Fucα1-2Galβ1-4GlcNAcβ1-6)GalNAco1, Galβ1-3[Fucα1-2(GalNAcα1-3)Galβ1-4GlcNAcβ1-6]GalNAco1, GlcNAcα1-4Galβ1-3(Fucα1-2Galβ1-4GlcNAcβ1-6)GalNAco1, Fucα1-2Galβ1-3(GlcNAcα1-4Galβ1-4GlcNAcβ1-6)GalNAco1, Fucα1-2Galβ1-4(6S)GlcNAcβ1-3(Galβ1-4GlcNAcβ1-6)Gal minus H2O, Fucα1-2Galβ1-3[Fucα1-2Galβ1-4(6S)GlcNAcβ1-6]GalNAco1, GlcNAcα1-4Galβ1-3(GlcNAcα1-4Galβ1-4GlcNAcβ1-6)GalNAco1, Ga1-GlcNAcβ1-3(GlcNAcα1-4Galβ1-4GlcNAcβ1-6)GalNAco1, GalNAcα1-3(Fucα1-2)Galβ1-4(6S)GlcNAcβ1-3Galβ1-3GalNAco1, Fucα1-2Galβ1-4GlcNAcβ1-3Galβ1-4GlcNAcβ1-3Galβ1-3GalNAco1, GlcNAcα1-4Galβ1-3[GalNAcα1-3(Fucα1-2)Galβ1-4GlcNAcβ1-6]GalNAco1, linear structure, Fucα1-2Galβ1-4(6S)GlcNAcβ1-3Galβ1-4GlcNAcβ1-3Galβ1-4GlcNAco1, and Fucα1-2Galβ1-4(6S)GlcNAcβ1-3Galβ1-4GlcNAcβ1-3(Galβ1-4GlcNAcβ1-6)Gal minus H2O.

In some embodiments, the mixture of free glycans and glycopeptides comprises glycopeptide-bound oligosaccharides having at least 10, at least 20, at least 50, at least 75, or all 99 of the structures provided above (e.g., the composition comprises at least 10, 20, 50, 75, or 99 glycopeptides, wherein each of the glycopeptides comprises a different glycopeptide-bound oligosaccharide from the above list). In some embodiments, the mixture of free glycans and glycopeptides comprises glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all 99 of the structures provided above.

In some embodiments, the therapeutic composition has a salt content of less than between about 5% and 1%. In some embodiments, the therapeutic composition has a salt content of less than about 2%. In some embodiments, the therapeutic composition has a salt content of less than about 1%.

The therapeutic composition may take the form of a slurry, powder, or liquid. In some embodiments, the therapeutic composition is a powder.

In some embodiments, the therapeutic composition does not comprise more than about 5%, more than about 4%, more than about 3%, more than about 2%, more than about 1%, more than about 0.5%, or more than about 0.1% free glycans by weight. In some embodiments, the therapeutic composition has a free glycan content of less than 1% by weight. In some embodiments, the therapeutic composition has a free glycan content of less than 0.1% by weight. In some embodiments, the therapeutic composition has a free glycan content of substantially zero. The phrase “free glycans” refers to glycans that are not attached to a protein or polypeptide.

The disease is not limited and may be any disease having a site with pathological neutrophil infiltration or pathological levels of neutrophils (e.g., a level of neutrophils causing tissue damage). In some embodiments, the disease is selected from the group consisting of excessive neutrophil infiltration of an organ or tissue, excessive neutrophil activation, acute lung injury, sepsis, ischemia-reperfusion injury, myocardial infarction, stroke, inflammatory bowel disease, acute colitis, ulcerative colitis, nonalcoholic fatty liver disease (NAFLD), metabolic-associated fatty liver disease (MAFLD), pleurisy, pulmonary fibrosis, systemic sclerosis, chronic obstructive pulmonary disease (COPD), adult respiratory response syndrome (ARDS), peripheral blood neutrophilia, systemic inflammatory response syndrome (SIRS), derangement of coagulation, fibrinolysis, disseminated intravascular coagulation, coagulopathy, vasculitis, or neutrophil-mediated tissue damage and remodelling. In some embodiments, the disease is nonalcoholic fatty liver disease (NAFLD) or metabolic-associated fatty liver disease (MAFLD).

Routes of administering the therapeutic composition are not limited and may be any suitable route. In some embodiments, the therapeutic composition is administered orally, parenteral, or rectally.

In some embodiments, the therapeutic composition is in the form of a pharmaceutical composition. In some embodiments, the therapeutic composition further comprises one or more excipients or carriers. In some embodiments, the therapeutic composition comprises a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients (i.e., the glycopeptides). The term “compatible”, as used herein, means that the components of the pharmaceutical compositions are capable of being comingled with an agent(i.e., the glycopeptides), and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

### Methods of Manufacture

Some aspects of the present disclosure are directed to a method of manufacturing a therapeutic composition disclosed herein, comprising the following steps: hydrolyzing an aqueous solution containing mucin powder with Ca(OH)2 at 60° C. for about 3 hours; cooling the solution to room temperature, adding diatomaceous earth, filtering the solution and collecting the filtrate; neutralizing the filtrate to a pH of 7.5 with CO2 and, optionally, adding diatomaceous earth, filtering and collecting the filtrate; contacting the filtrate with Dowex ion exchange hydrogen form resin to adjust the pH to 6.5 to 7, filtering and collecting the filtrate; and concentrating and spray drying the filtrate to obtain a powder. In some embodiments, the therapeutic composition is manufactured by a method comprising the method provided in Example 1 or 2 below.

In some embodiments, the method of manufacture comprises adding 20 g of ali mucin powder to 3.2 g of calcium hydroxide dissolved in 300 ml D.I. water, and stirring the same for 30 minutes at room temperature, followed by stirring the same at a temperature of 60° C. for about 3 hours. In some embodiments, the method of manufacture further comprises cooling the mixture to room temperature, adding about 3 g of Celite (diatomaceous earth), stirring for 10 minutes, and filtering with a Whatman paper filter and collecting the filtrate. In some embodiments, the method of manufacture further comprises neutralizing the filtrate using CO₂ to a pH of less than 7.5, then adding 1-2 g of Celite to the filtrate, stirring for 5 minutes, and filtering with a Whatman paper filter and collecting the filtrate. In some embodiments, the method of manufacture further comprises adding Dowex ion exchange hydrogen form resin to adjust the pH to 6.5 to 7, and filtering with a Whatman paper filter and collecting the filtrate. In some embodiments, the method of manufacture further comprises concentrating the filtrate by rotary evaporation followed by spray drying.

In some embodiments, the method of manufacture is a method of manufacture for GBX101 provided in WO 2020/157321, published Aug. 6, 2020, incorporated herein by reference in its entirety.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1—Preparation of GBX (i.e., GBX101)

### Protocol 1

300 ml D.I. water was introduced to a 500 ml beaker. Then, under magnetic stirring (500 rpm), 3.2 g of calcium hydroxide was added to the beaker. The suspension solution was kept at room temperature (about 25° C.) and under magnetic stirring (500 rpm) for 10 minutes.

Under magnetic stirring (800 rpm) and at room temperature (about 25° C.), 20 g of mucin was added to the suspension. The suspension was kept at room temperature (about 25° C.) and under magnetic stirring (800 rpm) for 30 minutes. The temperature was brought up to 60° C. using a heating plate. The reaction mixture was kept at 60° C. and under magnetic stirring (500 rpm) for 3 hours.

The reaction mixture was cooled down to room temperature. Then, about 3 g of Celite (diatomaceous earth) was added to the mixture under magnetic stirring (500 rpm) at room temperature (about 25° C.) for 10 minutes. The reaction mixture was then filtered under vacuum on a Büchner funnel with a paper filter (Whatman). The filtrate was neutralized to pH under 7.5 using CO₂. Then 1 to 2 g of celite was added to the filtrate. The suspension solution was stirring magnetically at 500 rpm for 5 minutes.

The suspension was filtered again under vacuum on a Buchner funnel with a paper filter (Whatman). Dowex ion exchange hydrogen form resin was added to adjust the pH to 6.5 to 7. Dowex ion exchange hydrogen form resin was filtered out under vacuum using a Buchner funnel with a paper filter (Whatman).

Finally, the filtrate was concentrated under rotary evaporator and then spray dried.

GBX101 was collected with a yield of about 50%.

The resulting GBX composition was found to have a total oligosaccharide content of >10% (w/w) with a ratio of glycopeptides:free glycans of greater than 4:1 (w/w). The total glycoprotein of the composition was 12% or less (w/w).

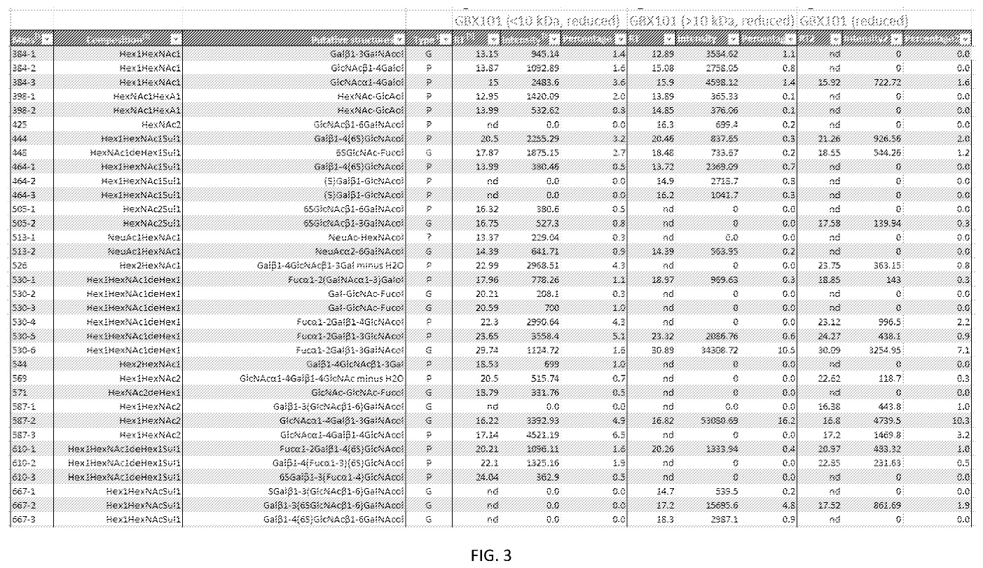

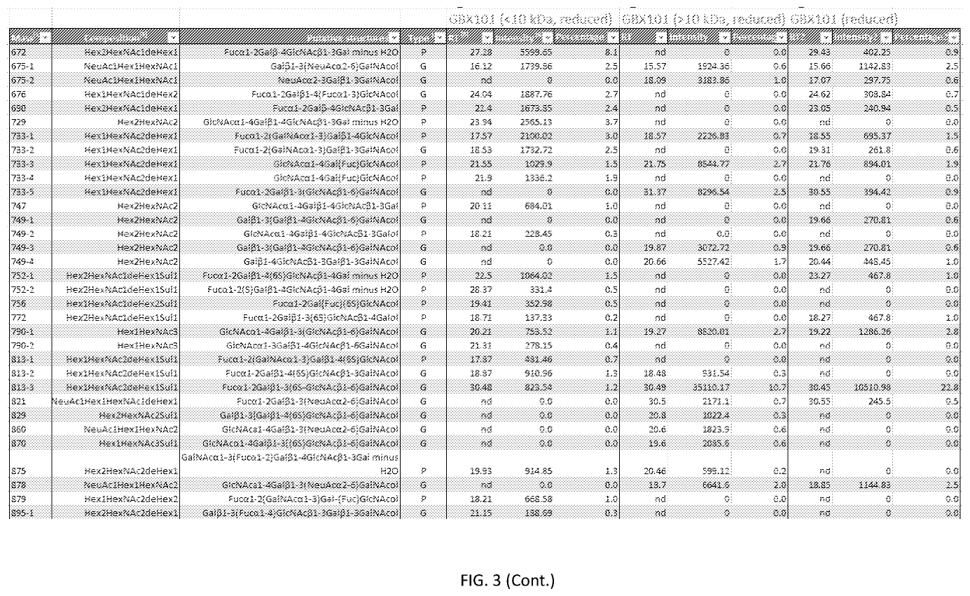

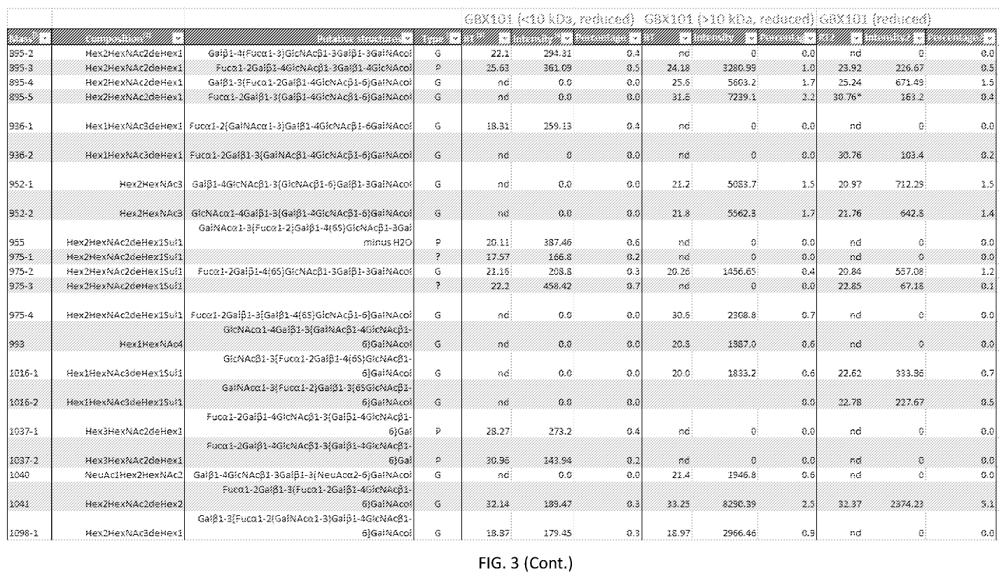

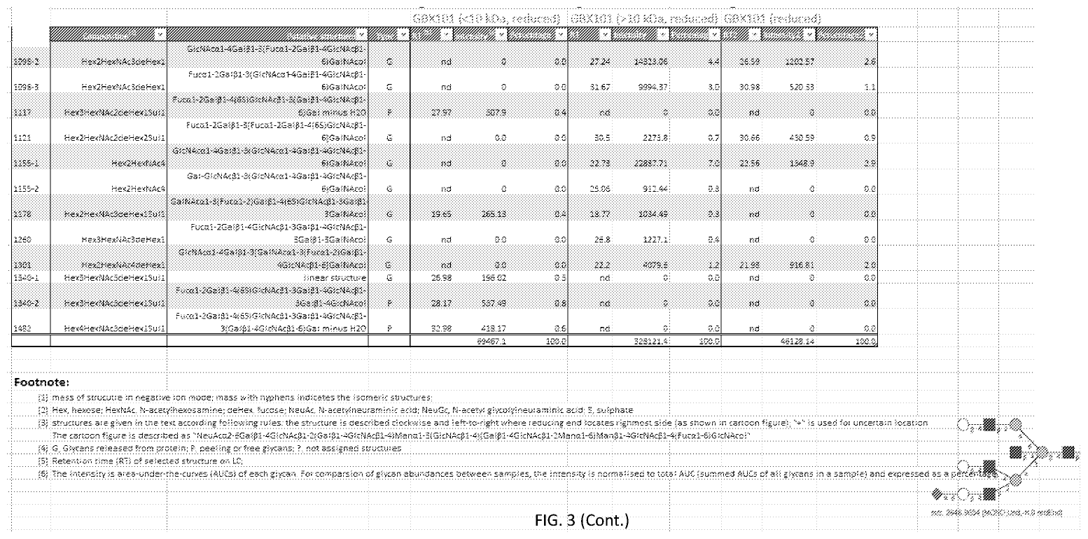

The bound glycans and the free glycans in the GBX composition were found to have the structures shown in FIG. 3.

### Example 2—Alternate GBX Purification Protocol

Protocol 2 (CO₂ neutralization omitted as Dowex ion exchange hydrogen resin can also remove the calcium)

300 ml D.I. water was introduced to a 500 ml beaker. Then, under magnetic stirring (500 rpm), 3.2 g of calcium hydroxide was added to the beaker. The suspension solution was kept at room temperature (about 25° C.) and under magnetic stirring (500 rpm) for 10 minutes.

Under magnetic stirring (800 rpm) and at room temperature (about 25° C.), 20 g of ali mucin (Porcine Gastric Mucin) was added to the suspension. The suspension was kept at room temperature (about 25° C.) and under magnetic stirring (800 rpm) for 30 minutes. The temperature was brought up to 60° C. using a heating plate. The reaction mixture was kept at 60° C. and under magnetic stirring (500 rpm) for 3 hours.

The reaction mixture was cooled down to room temperature. Then, about 3 g of Celite was added to the mixture under magnetic stirring (500 rpm) at room temperature (about 25° C.) for 10 minutes. The reaction mixture was then filtered under vacuum on a Buchner funnel with a paper filter (Whatman).

Dowex ion exchange hydrogen form resin was added to adjust the pH to 6.5 to 7. Dowex ion exchange hydrogen form resin was filtered out under vacuum using a Buchner funnel with a paper filter (Whatman).

Finally, the filtrate was concentrated under rotary evaporator and then spray dried.

GBX101 was collected with a yield of about 50%.

### Example 3—GBX Treatment of Acute Colitis

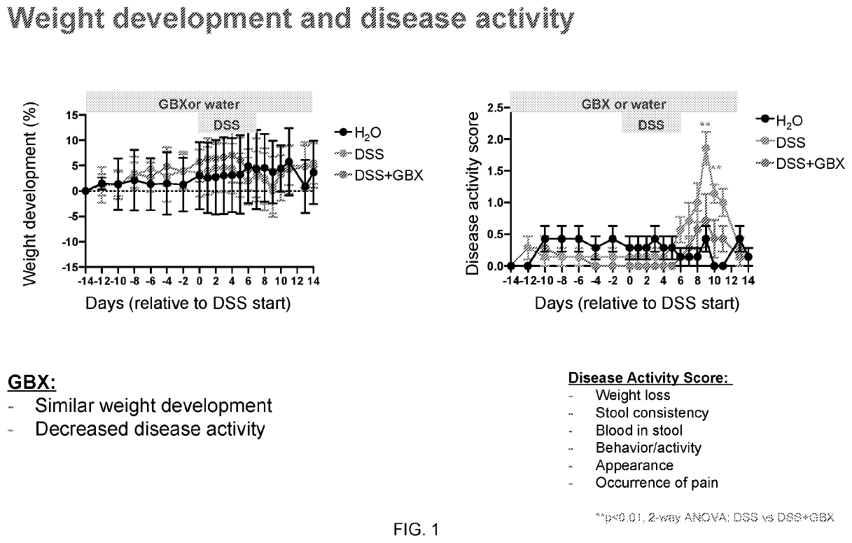

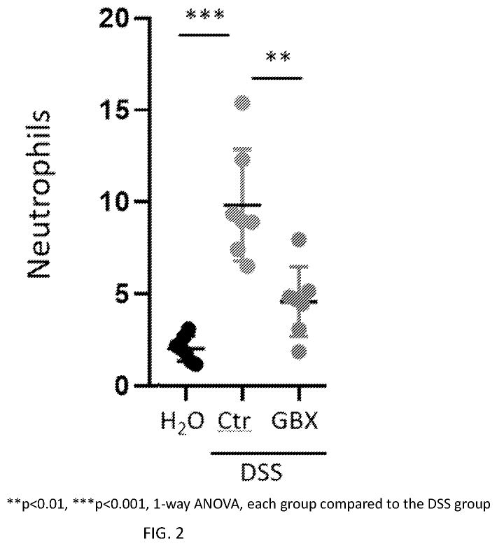

C57BL/6 mice (N=7/group) were treated either with normal drinking water or 3% GBX (i.e., GBX101) throughout the experiment. To prevent bacterial growth, the drinking water/drinking water with GBX was filtered every day and replaced every other day. After 14 days of pre-treatment, acute colitis was induced by adding 1.5% DSS to the drinking water or 3% GBX for 7 days. After DSS-induction, the drinking water was replaced by normal drinking water or 3% GBX and mice left to recover for 7 days. At the last day of the experiment, mice were anesthetized, subjected to mouse endoscopy prior to blood and organ collection. Colon length, spleen weight, endoscopic and histologic scores of the colon, and flow cytometric analysis of immune cell composition in colon, mesenteric lymph nodes and spleen were used to determine colitis severity. These results are shown in FIG. 1. Neutrophils were determined as CD45+, CD3−, B220−, F4/80−, Gr1+, CD11b+ cells as shown in FIG. 2.

What is claimed is:

1. A method of reducing neutrophils or neutrophil infiltration at the site of a disease in a subject in need thereof comprising administering a therapeutic composition comprising a mixture of free glycans and glycopeptides obtained from gastrointestinal tract mucins, wherein:
a. the composition is obtained without subjecting the mucins or a partially purified fraction thereof to conditions or reagents that cause complete release of oligosaccharides from glycoproteins or glycopeptides;
b. the total oligosaccharide content of the composition is >10% (w/w);
c. the ratio of glycopeptides:free glycans is >4:1 (w/w); and
d. the total glycoprotein content of the composition is 12% or less (w/w).

2. The method of claim 1, wherein the mixture of free glycans and glycopeptides comprises at least one glycopeptide-bound oligosaccharide having a general formulae selected from Hex1HexNAc1, Hex1HexNAc1, Hex1HexNAc1, HexNAc1HexA1, HexNAc1HexA1, HexNAc2, Hex1HexNAc1Sul1, HexNAc1deHex1Sul1, Hex1HexNAc1Sul1, Hex1HexNAc1Sul1, Hex1HexNAc1Sul1, HexNAc2Sul1, HexNAc2Sul1, NeuAc1HexNAc1, NeuAclHexNAc1, Hex2HexNAc1, Hex1HexNAc1deHex1, Hex1HexNAc1deHex1, Hex1HexNAc1deHex1, Hex1HexNAc1deHex1, Hex1HexNAc1deHex1, Hex1HexNAc1deHex1, Hex2HexNAc1, Hex1HexNAc2, HexNAc2deHex1, Hex1HexNAc2, Hex1HexNAc2, Hex1HexNAc2, Hex1HexNAc1deHex1Sul1, Hex1HexNAc1deHex1Sul1, Hex1HexNAc1deHex1Sul1, Hex1HexNAcSul1, Hex1HexNAcSul1, Hex1HexNAcSul1, Hex2HexNAc1deHex1, NeuAc1Hex1HexNAc1, NeuAc1Hex1HexNAc1, Hex1HexNAc1deHex2, Hex2HexNAc1deHex1, Hex2HexNAc2, Hex1HexNAc2deHex1, Hex1HexNAc2deHex1, Hex1HexNAc2deHex1, Hex1HexNAc2deHex1, Hex1HexNAc2deHex1, Hex2HexNAc2, Hex2HexNAc2, Hex2HexNAc2, Hex2HexNAc2, Hex2HexNAc2, Hex2HexNAc1deHex1Sul1, Hex2HexNAc1deHex1Sul1, Hex1HexNAc1deHex2Sul1, Hex2HexNAc1deHex1Sul1, Hex1HexNAc3, Hex1HexNAc3, Hex1HexNAc2deHex1Sul1, Hex1HexNAc2deHex1Sul1, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, NeuAc1Hex1HexNAc2, Hex1HexNAc2deHex2, Hex2HexNAc2deHex1, Hex2HexNAc2deHex1, Hex2HexNAc2deHex1, Hex2HexNAc2deHex1, Hex2HexNAc2deHex1, Hex1HexNAc3deHex1, Hex1HexNAc3deHex1, Hex2HexNAc3, Hex2HexNAc3, Hex2HexNAc2deHex1Sul1, Hex2HexNAc2deHex1Sul1, Hex2HexNAc2deHex1Sul1, Hex2HexNAc2deHex1Sul1, Hex2HexNAc2deHex1Sul1, Hex1HexNAc4, Hex1HexNAc3deHex1Sul1, Hex1HexNAc3deHex1Sul1, Hex3HexNAc2deHex1, Hex3HexNAc2deHex1, NeuAc1Hex2HexNAc2, Hex2HexNAc2deHex2, Hex2HexNAc3deHex1, Hex2HexNAc3deHex1, Hex2HexNAc3deHex1, Hex3HexNAc2deHex1Sul1, Hex2HexNAc2deHex2Sul1, Hex2HexNAc4, Hex2HexNAc4, Hex2HexNAc3deHex1Sul1, Hex3HexNAc3deHex1, Hex2HexNAc4deHex1, Hex3HexNAc3deHex1Sul1, Hex3HexNAc3deHex1Sul1, and Hex4HexNAc3deHex1Sul1.

3. The method of claim 2, wherein the mixture of free glycans and glycopeptides comprises glycopeptide-bound oligosaccharides having at least 10, at least 20, at least 50, at least 75, or all 99 of the general formulae.

4. The method of claims 1-3, wherein the mixture of free glycans and glycopeptides comprises at least one glycopeptide-bound oligosaccharide having a structure selected from Galβ1-3GalNAco1, GlcNAcβ1-4Galo1, GlcNAcα1-4Galo1, HexNAc-GlcAo1, HexNAc-GlcAo1, GlcNAcβ1-6GalNAco1, Galβ1-4(6S)GlcNAco1, 6SGlcNAc-Fuco1, Galβ1-4(6S)GlcNAco1, (S)Galβ1-GlcNAco1, (S)Galβ1-GlcNAco1, 6SGlcNAcβ1-6GalNAco1, 6SGlcNAcβ1-3GalNAco1, NeuAc-HexNAco1, NeuAcα2-6GalNAco1, Galβ1-4GlcNAcβ1-3Gal minus H2O, Fucα1-2(GalNAcα1-3)Galo1, Ga1-GlcNAc-Fuco1, Ga1-GlcNAc-Fuco1, Fucα1-2Galβ1-4GlcNAco1, Fucα1-2Galβ1-3GlcNAco1, Fucα1-2Galβ1-3GalNAco1, Galβ1-4GlcNAcβ1-3Gal, GlcNAcα1-4Galβ1-4GlcNAc minus H2O, GlcNAc-GlcNAc-Fuco1, Galβ1-3(GlcNAcβ1-6)GalNAco1, GlcNAcα1-4Galβ1-3GalNAco1, GlcNAcα1-4Galβ1-4GlcNAco1, Fucα1-2Galβ1-4(6S)GlcNAco1, Galβ1-4(Fucα1-3)(6S)GlcNAco1, 6SGalβ1-3(Fucα1-4)GlcNAco1, SGalβ1-3(GlcNAcβ1-6)GalNAco1, Galβ1-3(6SGlcNAcβ1-6)GalNAco1, Galβ1-4(6S)GlcNAcβ1-6GalNAco1, Fucα1-2Galβ-4GlcNAcβ1-3Gal minus H2O, Galβ1-3(NeuAcα2-6)GalNAco1, NeuAcα2-3Galβ1-3GalNAco1, Fucα1-2Galβ1-4(Fucα1-3)GlcNAco1, Fucα1-2Galβ-4GlcNAcβ1-3Gal, GlcNAcα1-4Galβ1-4GlcNAcβ1-3Gal minus H2O, Fucα1-2(GalNAcα1-3)Galβ1-4GlcNAco1, Fucα1-2(GalNAcα1-3)Galβ1-3GalNAco1, GlcNAcα1-4Gal(Fuc)GlcNAco1, GlcNAcα1-4Gal(Fuc)GlcNAco1, Fucα1-2Galβ1-3(GlcNAcβ1-6)GalNAco1, GlcNAcα1-4Galβ1-4GlcNAcβ1-3Gal, Galβ1-3(Galβ1-4GlcNAcβ1-6)GalNAco1, GlcNAcα1-4Galβ1-4GlcNAcβ1-3Galo1, Galβ1-3(Galβ1-4GlcNAcβ1-6)GalNAco1, Galβ1-4GlcNAcβ1-3Galβ1-3GalNAco1, Fucα1-2Galβ1-4(6S)GlcNAcβ1-4Gal minus H2O, Fucα1-2(S)Galβ1-4GlcNAcβ1-4Gal minus H2O, Fucα1-2Gal(Fuc)(6S)GlcNAco1, Fucα1-2Gal(31-3(6S)GlcNAcβ1-4Galo1, GlcNAcα1-4Galβ1-3(GlcNAcβ1-6)GalNAco1, GlcNAcα1-3Galβ1-4GlcNAcβ1-6GalNAco1, Fucα1-2(GalNAcα1-3)Galβ1-4(6S)GlcNAco1, Fucα1-2Galβ1-4(6S)GlcNAcβ1-3GalNAco1, Fucα1-2Galβ1-3(6S-GlcNAcβ1-6)GalNAco1, Fucα1-2Galβ1-3(NeuAcα2-6)GalNAco1, Galβ1-3[Galβ1-4(6S)GlcNAcβ1-6]GalNAco1, GlcNAcα1-4Galβ1-3(NeuAcα2-6)GalNAco1, GlcNAcα1-4Galβ1-3[(6S)GlcNAcβ1-6]GalNAco1, GalNAcα1-3(Fucα1-2)Galβ1-4GlcNAcβ1-3Gal minus H2O, GlcNAcα1-4Galβ1-3(NeuAcα2-6)GalNAco1, Fucα1-2(GalNAcα1-3)Ga1-(Fuc)GlcNAco1, Galβ1-3(Fucα1-4)GlcNAcβ1-3Galβ1-3GalNAco1, Galβ1-4(Fucα1-3)GlcNAcβ1-3Galβ1-3GalNAco1, Fucα1-2Galβ1-4GlcNAcβ1-3Galβ1-4GlcNAco1, Galβ1-3(Fucα1-2Galβ1-4GlcNAcβ1-6)GalNAco1, Fucα1-2Galβ1-3(Galβ1-4GlcNAcβ1-6)GalNAco1, Fucα1-2(GalNAcα1-3)Galβ1-4GlcNAcβ1-6GalNAco1, Fucα1-2Galβ1-3(GalNAcβ1-4GlcNAcβ1-6)GalNAco1, Galβ1-4GlcNAcβ1-3(GlcNAcβ1-6)Galβ1-3GalNAco1, GlcNAcα1-4Galβ1-3(Galβ1-4GlcNAcβ1-6)GalNAco1, GalNAcα1-3(Fucα1-2)Galβ1-4(6S)GlcNAcβ1-3Gal minus H2O, Fucα1-2Galβ1-4(6S)GlcNAcβ1-3Galβ1-3GalNAco1, Fucα1-2Galβ1-3[Galβ1-4(6S)GlcNAcβ1-6]GalNAco1, GlcNAcα1-4Galβ1-3(GalNAcβ1-4GlcNAcβ1-6)GalNAco1, GlcNAcβ1-3[Fucα1-2Galβ1-4(6S)GlcNAcβ1-6]GalNAco1, GalNAcα1-3(Fucα1-2)Galβ1-3(6SGlcNAcβ1-6)GalNAco1, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAco1, Fucα1-2Galβ1-3(Fucα1-2Galβ1-4GlcNAcβ1-6)GalNAco1, Galβ1-3[Fucα1-2(GalNAcα1-3)Galβ1-4GlcNAcβ1-6]GalNAco1, GlcNAcα1-4Galβ1-3(Fucα1-2Galβ1-4GlcNAcβ1-6)GalNAco1, Fucα1-2Galβ1-3(GlcNAcα1-4Galβ1-4GlcNAcβ1-6)GalNAco1, Fucα1-2Galβ1-4(6S)GlcNAcβ1-3(Galβ1-4GlcNAcβ1-6)Gal minus H2O, Fucα1-2Galβ1-3[Fucα1-2Galβ1-4(6S)GlcNAcβ1-6]GalNAco1, GlcNAcα1-4Galβ1-3(GlcNAcα1-4Galβ1-4GlcNAcβ1-6)GalNAco1, Ga1-GlcNAcβ1-3(GlcNAcα1-4Galβ1-4GlcNAcβ1-6)GalNAco1, GalNAcα1-3(Fucα1-2)Galβ1-4(6S)GlcNAcβ1-3Galβ1-3GalNAco1, Fucα1-2Galβ1-4GlcNAcβ1-3Galβ1-4GlcNAcβ1-3Galβ1-3GalNAco1, GlcNAcα1-4Galβ1-3[GalNAcα1-3(Fucα1-2)Galβ1-4GlcNAcβ1-6]GalNAco1, linear structure, Fucα1-2Galβ1-4(6S)GlcNAcβ1-3Galβ1-4GlcNAcβ1-3Galβ1-4GlcNAco1, and Fucα1-2Galβ1-4(6S)GlcNAcβ1-3Galβ1-4GlcNAcβ1-3(Gal 31-4GlcNAcβ1-6)Gal minus H2O.

5. The method of claim 4, wherein the mixture of free glycans and glycopeptides comprises glycopeptide-bound oligosaccharides having at least 10, at least 20, at least 50, at least 75, or all 99 of the structures.

6. The method of claims 1-5, wherein the therapeutic composition has a salt content of less than about 2%.

7. The method of claims 1-6, wherein the therapeutic composition is a powder.

8. The method of claims 1-7, wherein the therapeutic composition has a free glycan content of less than 0.1% by weight.

9. The method of claims 1-8, wherein the disease is selected from the group consisting of excessive neutrophil infiltration of an organ or tissue, excessive neutrophil activation, acute lung injury, sepsis, ischemia-reperfusion injury, myocardial infarction, stroke, nonalcoholic fatty liver disease (NAFLD), metabolic-associated fatty liver disease (MAFLD), pleurisy, pulmonary fibrosis, systemic sclerosis, chronic obstructive pulmonary disease (COPD), adult respiratory response syndrome (ARDS), peripheral blood neutrophilia, systemic inflammatory response syndrome (SIRS), derangement of coagulation, fibrinolysis, disseminated intravascular coagulation, coagulopathy, vasculitis, or neutrophil-mediated tissue damage and remodelling.

10. The method of claims 1-9, wherein the therapeutic composition is administered orally or rectally.

11. The method of claims 1-10, wherein the therapeutic composition further comprises one or more excipients or carriers.

12. A method of manufacturing the therapeutic composition of claims 1-11, comprising the following steps:
a. hydrolyzing an aqueous solution containing mucin powder with Ca(OH)2 at 60° C. for about 3 hours;
b. cooling the solution to room temperature, adding diatomaceous earth, filtering the solution and collecting the filtrate;
c. neutralizing the filtrate to a pH of 7.5 with CO₂ and, optionally, adding diatomaceous earth, filtering and collecting the filtrate;
d. contacting the filtrate with Dowex ion exchange hydrogen form resin to adjust the pH to 6.5 to 7, filtering and collecting the filtrate; and
e. concentrating and spray drying the filtrate to obtain a powder.
